(19) United States
(12) Patent Application Publication
(10) Pub. No.: US 2023/0174928 A1
(43) Pub. Date: Jun. 8, 2023

(54) MULTI-LAYER NANOFIBER SCAFFOLDS

(71) Applicant: University of Georgia Research Foundation, Inc., Athens, GA (US)

(72) Inventors: Nataraja S. Yadavalli, Athens, GA (US); Darya Asheghali, Durham, NC (US); Sergiy Minko, Athens, GA (US); Alexander Tokarev, Mt Pleasant, SC (US); Vladimir Reukov, Athens, GA (US); Brianna Blevins, Rising Fawn, GA (US); Ummay Mowshome Jahan, Raleigh, NC (US)

(21) Appl. No.: 18/105,350

(22) Filed: Feb. 3, 2023

Related U.S. Application Data
(63) Continuation-in-part of application No. 17/169,940, filed on Feb. 8, 2021.
(60) Provisional application No. 62/971,512, filed on Feb. 7, 2020.
(60) Provisional application No. 63/267,506, filed on Feb. 3, 2022.

(51) Int. Cl. C12N 5/00; B33Y 80/00; B82Y 30/00; A23J 3/22

(52) U.S. Cl. CPC C12N 5/0068; A23J 3/227; B33Y 80/00; B82Y 30/00; C12N 2513/00; C12N 2533/00

(57) ABSTRACT

Provided are 3D cell culture scaffolds, 3D nanofiber scaffolds, and edible 3D nanofiber scaffolds for cultured meat. Described is a 3D cell culture scaffold including a plurality of laminated nanofiber layers. Each layer is formed by an array of nanofibers. The diameter of each of the nanofibers in the arrays can have a tunable, predetermined diameter and can be formed from materials including a natural polymer, a synthetic polymer, a biocompatible material, or a combination thereof. Each of the nanofibers in the arrays can have controlled alignment, angle, and spacing from one another. The layers can be spaced by spacer fibers or spacer sheets. The scaffold can have a porosity of about 50% to 99%. Edible 3D scaffolds for cultured meat are also provided where the nanofibers and spacers are edible.

### CROSS REFERENCE TO RELATED APPLICATIONS

This application is a Continuation-in-Part of application having serial number 17/169,940, filed on Feb. 8, 2021, which application claims priority to U.S. Provisional Application 62/971,512 filed on Feb. 7, 2020. This application also claims priority to U.S. Provisional Application entitled “MULTI-LAYER NANOFIBER SCAFFOLDS,” having serial number 63/267,506 filed on Feb. 3, 2022, each of which are entirely incorporated herein by reference.

### BACKGROUND

Three-dimensional cell culture scaffolds (3DCC) and 3D nanofiber scaffolds (3DFS) are highly desired for adherent cell culture research and development in tissue engineering, regenerative medicine (RM) specifically cell-based therapeutics and cell manufacturing for bioprinting, cell-cultured meat (CM), and cultured seafood (CS) applications. Each platform is tuned to support specific cell types. Considering the complexity of cellular physiology in the tissue microenvironment and multitude of growth parameters, often these platforms are not widely applicable, and the results are not reproducible. A major deficit of current solutions is poor scalability of these platforms for cell therapy and cell-cultured meat applications. They are also largely restricted to laboratory usage due to the limitations of the underlying technologies. There is a need to standardize the 3DCC platforms for a wide adaptation in research and in industrial scale adherent cell manufacturing for RM and CM in lieu of 2D cell culture (2DCC) and non-adherent techniques.

### SUMMARY

Embodiments of the present disclosure provide for 3D cell culture scaffolds, 3D nanofiber scaffolds, edible 3D nanofiber scaffolds and the like.

An embodiment of the present disclosure includes a 3D cell culture scaffold that includes a plurality of laminated nanofiber layers. Each layer is formed by an array of nanofibers. The diameter of each of the nanofibers in the arrays can have a tunable, predetermined diameter of about 50 nm to 100 µm. The nanofibers can be formed from materials including a natural polymer, a synthetic polymer, a biocompatible material, or a combination thereof. Each of the nanofibers in the arrays can have controlled alignment, angle, and spacing from one another. The scaffold can have a porosity of about 50% to 99%.

An embodiment of the present disclosure also includes an edible 3D scaffold for cultured meat. The edible scaffold can include a plurality of edible nanofibers in nanofiber layers laminated together. Each layer is formed by an array of nanofibers, wherein each of the nanofibers in the arrays has a predetermined diameter of about 50 nm to 100 µm. The diameter of individual nanofibers can be controlled. The nanofibers in the arrays also have controlled alignment, angle, and spacing from one another. The scaffold can have a porosity of about 50% to 99%.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure.

FIGS. 1A and 1B are camera images of two views of an example of a touch spun device (configuration 1) for rapid nanofiber fabrication and 3D fiber scaffolding in accordance with embodiments of the present disclosure. Here, a rapidly rotating carousel with fiber drawing rod is rotated around the fiber positioning/collecting bed (dashed arrows indicate rotation).

FIG. 2A is a schematic illustration of the overall touch spinning principle. FIG. 2B is camera image of a touch spun device (configuration 2). In the rapid fiber spinning prototype shown, fibers are drawn from a polymer solution extruding from a needle and spun around a drum (also referred to herein as a collection frame). FIG. 2C is a camera image of the drum in FIG. 2B. An arrow in FIG. 2C indicates the single layers of nanofibers spun around the drum. FIG. 2D shows controlled fiber alignment and spacing in two directions.

FIGS. 3A-3G illustrate examples of 3D cell culture systems using the 3D nanofiber scaffolds (3DFS) as described herein. FIG. 3A is a camera image of the 3DFS embedded inserts in a 6-well plate. FIG. 3B is an example of the insert design to embed the 3DFS. FIG. 3C is a diagram showing an example of large 3DFS scaffolding via PCL spacer-based edge welding and stacking (not to scale). FIGS. 3D-3G are camera images of a scaled 3DFS. FIG. 3D is a top image of a 11x11x1 cm³. FIG. 3E is a side view of the 3DFS in FIG. 3D. FIG. 3F is a close-up image of nanofiber layers with spacers indicated by the dashed rectangle in FIG. 3D. FIG. 3G is a camera image of a thermal welded flexible large scaffold such as the one in FIG. 3D without a cartridge.

FIGS. 4A-4C show precision aligned 3D nanofiber scaffold fabrication. FIGS. 4D-4G show scaffold engineering. FIG. 4A iis a scanning electron microscope image of single filament PCL nanofibers are used as building blocks to construct layer-by-layer precision aligned 2D scaffold arrays. FIG. 4B is a scanning electron microscope image demonstrating angular alignment of fibers (0° - 179°) achieved by the scaffolding devices described herein; spacer fibers are shown in the top figure, and the bottom figure is a magnified view of the spacer fibers showing the angles between them. FIG. 4C (top) is an SEM image corner view of 0.43 mm thick (area-1 cm²) 3D nanofiber scaffold (3DFS) and (bottom) with 100 ±5 µm spacing between nanofibers and 50 ±5 µm gap between layers. FIG. 4D shows a camera image of a 3DFS embedded in multi-well plate inserts. FIG. 4E is an illustration of 3DFS design for tunable porosity. FIG. 4F shows a 3DFS having controlled vasculature with ‘/’ µm (e.g., 200 ±5 µm) thick nanofiber zones for 3D cell culture and ‘d’ µm (e.g., 200 ±5 µm) gap for mass transport. FIG. 4G is a camera image of a of scalable 3DFS cartridge fabrication with 8 cm³ cube geometry, controlled porosity and layer spacing.

FIGS. 5A-5H show 3D cell culture growth on 3DFS inserts, showing static 3D cell culture studies on PCL 3DFS inserts in 6-well plate. FIGS. 5A-5F show 17-day old 3T3 fibroblast culture on 3DFS insert. FIGS. 5A and 5B are illustrations of 3DFS structures used in 3T3 fibroblast culture. FIG. 5A is built with 3 layers, thickness 0.2 mm. FIG. 5B built with 5 layers, thickness 0.5 mm. FIGS. 5C and 5D show calcein-AM staining. FIG. 5C shows confined fibroblast growth in individual layers of the three-layer 3DFS. FIG. 5D shows confined fibroblast growth in individual layers of five-layer 3DFS. FIG. 5E shows DAPI staining, demonstrating dense fibroblast growth in single layer of an 3DFS insert (e.g., insert as shown in FIG. 4D).

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, material science, and the like, which are within the skill of the art.

The following examples are put forth so as to provide those of ordinary skill in the art with a complete disclosure and description of how to perform the methods and use the devices disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Definitions

“Nanofiber” is a fiber having a radius on the nano-scale (e.g., 1 nm to 1000 nm).

“Polymer” is any natural or synthetic molecule that can form long molecular chains, such as polysaccharides, proteins, nylons, polyethylene, polystyrene, polylactide, polyglycolide, polypropylene, polyacetylene, polyphenylene vinylene, polypyrrole, polyesters, polyurethanes, polycaprolactone, polyethylene glycol, food compatible and edible biopolymers including, polyvinyl acetate, cellulose, methyl cellulose, cellulose acetate, chitosan, alginic acid, soy proteins, prolamine proteins, collagen, silk fibroin, combinations of these, and blends of these.

“Alignment” and “aligned” as used herein refers to a spatially reproduced fiber-fiber distance or layer-layer gap in three-dimensional nanofiber scaffolds. For example, alignment refers to a precise spacing and orientation between a pair of fibers in a predetermined, non-random manner, such nanofibers are ordered and precisely spaced from one another according to a particular need. For example, the distance between a pair of nanofibers is at a set distance (e.g., 10 µm to 100 µm, 10 µm to 20 µm) and/or at a desired angle (e.g., 0 to 179 degrees) relative to each other or to another fiber in the array.

“Orientation” as used herein refers to a collection of fibers in a specific direction without the controlled spatial position of fibers (e.g. when using electrospinning with a rotating collector drum, one can achieve all fibers coiled around the drum linearly). The electrospinning technique generally produces oriented fibers but not aligned fibers, despite common use of the term alignment/aligned in the industry to refer to fibers oriented in substantially the same direction but without precision spacing between fibers.

### General Discussion

In accordance with the purpose(s) of the present disclosure, as embodied and broadly described herein, embodiments of the present disclosure, in some aspects, relate to 3D nanofiber scaffolds, 3D cell culture scaffolds that include nanofibers, and methods of making 2D nanofiber arrays and 3D nanofiber scaffolds.

In general, embodiments of the present disclosure provide for 3D scaffolds and methods for making scaffolds that include nanofibers made by various fiber fabrication methods.

For the purposes of discussion, the 3D scaffolds described herein may be referred to as nanofiber scaffolds. The nanofiber scaffolds include fibers in the nanofiber diameter range. The scaffolds may also include larger fibers such as fibers having a diameter in the micron range.

3D nanofiber scaffolds - In general, the 3D nanofiber scaffolds can be formed from fibers generated by various fiber-forming methods including but not limited to gravitational drawing of a polymer solution, touch spinning, wet spinning, track spinning, or a combination thereof. The 3D nanofiber scaffolds are formed from layers of 3D nanofiber arrays having controlled fiber alignment, fiber orientation, fiber spacing, and fiber diameter.

The 3D nanofiber scaffolds are formed from layers of fiber arrays. In some embodiments, the layers can be formed by stacking of discrete arrays. The array layers can be separated by spacer fibers. The scaffold can be fabricated layer-by-layer from 2D arrays. In some embodiments, the arrays can be structured on a collection frame. The collection frame can be any desired shape and desired thickness. In other embodiments, the fibers themselves can provide structure to the array layer (e.g. by having thicker and/or denser fiber arrangement around the edges).

In other embodiments, the structure of the scaffold can be built by continuous arrays. Layers can be formed such as through folding, rotation, spiraling, etc. of the arrays.

The fibers can have diameters from about 50 nm to 100 µm, about 500 nm to 2 µm, or about 50 nm to 999 nm. In various embodiments, the scaffold can have a porosity of about 50% to 99%, about 70% to 99%, about 70 to 90%, about 90% to 99%, or about 95% to 99%.

Each nanofiber can be spaced and aligned in a predetermined, non-random manner, such that the resulting array of nanofibers is ordered and precisely spaced according to a particular need. For example, the distance between a pair of nanofibers is at a set distance (e.g., 10 µm to 100 µm, 10 µm to 20 µm) and/or at a desired angle (e.g., 0 to 179 degrees) relative to each other or to another fiber in the array. The fibers can be parallel to one another and/or in any desired angle and position relative to the first single filament nanofiber. In some embodiments, all of the nanofibers in a given array will all be parallel to one another. In some embodiments, the fibers can be placed at predetermined angles relative to one another or to the collection frame. In some embodiments, the fibers can be collected (e.g. non-randomly placed on the collection frame) such that they form crisscross or woven patterns in a given array. An alignment angle between any two adjacent single filament nanofibers can be independently selected from about 0 to 179 degrees, and a spacing between any two adjacent fibers can be independently selected from about 10 micron or larger. Each layer can be rotated in the X-Y plane orthogonal to a layer orientation or fiber orientation of an adjacent layer.

The ordered arrays of nanofibers arrays can be layered to form three-dimensional scaffolds that can be customized with varying fiber materials, diameters, elastic modulus, orientations, angles, spacing, spaces between layers, and geometries (e.g., flat sheet, tube, cylinder, cube, etc.).

The 3D nanofiber scaffolds described herein can be used for cell culturing and engineered meat culturing, as well as for other applications such as electronics and filtration.

3D cell culture scaffolds - The 3D nanofiber scaffolds provided herein can be 3D cell culture scaffolds. The cell culture scaffolds can be used for such as adherent cell culture research and development in tissue engineering, regenerative medicine (RM) including cell-based therapeutics and cell manufacturing for bioprinting, cell-cultured meat (CM), and cultured seafood (CS) applications. The cell culture scaffolds can be formed with specially designed gaps to allow for nutrient, gas and metabolite exchange. In one embodiment, the gaps can be about 200 µm). This type of cell culture scaffold can reduce necrosis in 3D cell aggregate and help the continuous growth of culture for weeks. Advantageously, commercially interesting metabolites (e.g., antibodies, exosomes, etc.) can be extracted efficiently. 3D cell culture scaffolds as described herein can be used to scale up the 3D cell culture techniques for industrial scale metabolite or cell-based therapeutics production as an alternative to bioreactors.

The present disclosure provides for 3D cell culture scaffolds that include at least two layers of fibers. Each layer can be formed by an array of fibers. The fibers can have diameters from about 50 nm to 100 µm, about 500 nm to 2 µm, or about 50 nm to 999 nm. Larger diameter fibers are formed to serve as spacer fibers, whereas smaller diameter fibers (e.g., less than 2 microns) are used for cell anchoring and proliferation. The fibers can include an edible and/or biocompatible polymer (e.g., conductive to enhanced cell attachment and growth, non-cytotoxic, and do not solicit an immune response) suitable for using as is or surface functionalization with proteins or biochemical cues post scaffolding. In various embodiments, the scaffold can have a porosity of about 50% to 99%, about 70% to 99%, or about 90% to 99%. Typically, cell diameter varies (e.g., 5 µm, 20 µm, 150 µm etc.) depending on the morphology and the cell culture environment. Accordingly, for cell culture scaffolds the fiber diameter will be smaller than the cell diameter so that the fiber surface does not act like a flat surface.

Another problem is, fiber over 10 µm means, fiber material share of the overall scaffold volume is increasing and space for cell growth decreasing. For any application this is not good. Sometimes, cells prefer really nanofibers at around 1 µm or below in diameter.

Issues with existing 3D cell culture technologies include poor nutrient, gas, and metabolite exchange between the extracellular matrix and external environment with culture media. This can result in 3D cell necrosis causing workflow problems and costing time and resources. Advantageously, the 3D cell culture scaffolds described herein can be formed with over 90% free volume (e.g., porosity) and uninterrupted, predesigned micro-perfusion channels that allow for nutrient, gas and metabolite exchange until the 3D cell culture is harvested. The nanofiber spacing and alignment of each fiber can be precisely controlled during manufacture such that the fiber layer-layer spacing forms microvascular mimetic channels according to need. The micro-perfusion channels are formed by the spacer fibers between the nanofiber arrays. The size, shape, and placement of these channels can be customized by tuning the spacer fibers. These channels can be designed and easily incorporated into any 3D scaffold as a built-in structural feature to achieve media, gas, and metabolite exchange in and out of the scaffold as well as for cell diffusion uniformly throughout the 3D scaffold.

In various embodiments, the 3D cell culture scaffolds can be formed on frames of about 1×1 cm² to 15×15 cm² dimensions for laboratory scale research applications, which allow the scaffolds to fit in standard cell culture flasks. Further, the 3D fiber scaffolded frames can be embedded in a cup-like insert to fit into standard multi-well cell culture plates, or other insert shapes to fit into petri dishes and T-flasks for cell culture and as cartridges in bioreactor systems. Scaffolds larger than 15×10×2 cm³ are suitable for commercial size scale-up needs and can be designed as a stack of scaffold frames to fit into bioreactor style containers with continuous circulation of all exchange materials (e.g., nutrients, gas, metabolites, etc.). The applications of these scaffolds include but are not limited to 3D cell cultures and tissue engineering. In some embodiments, the scaffolds can have a total geometric volume of about 0.125 cm³ to 5000 cm³.

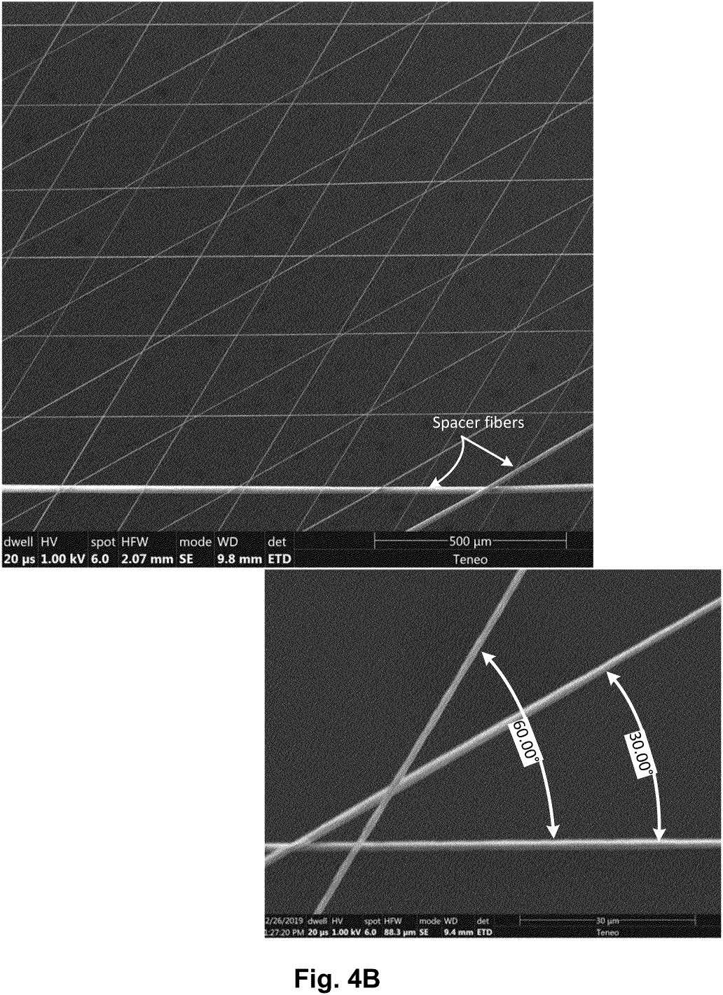

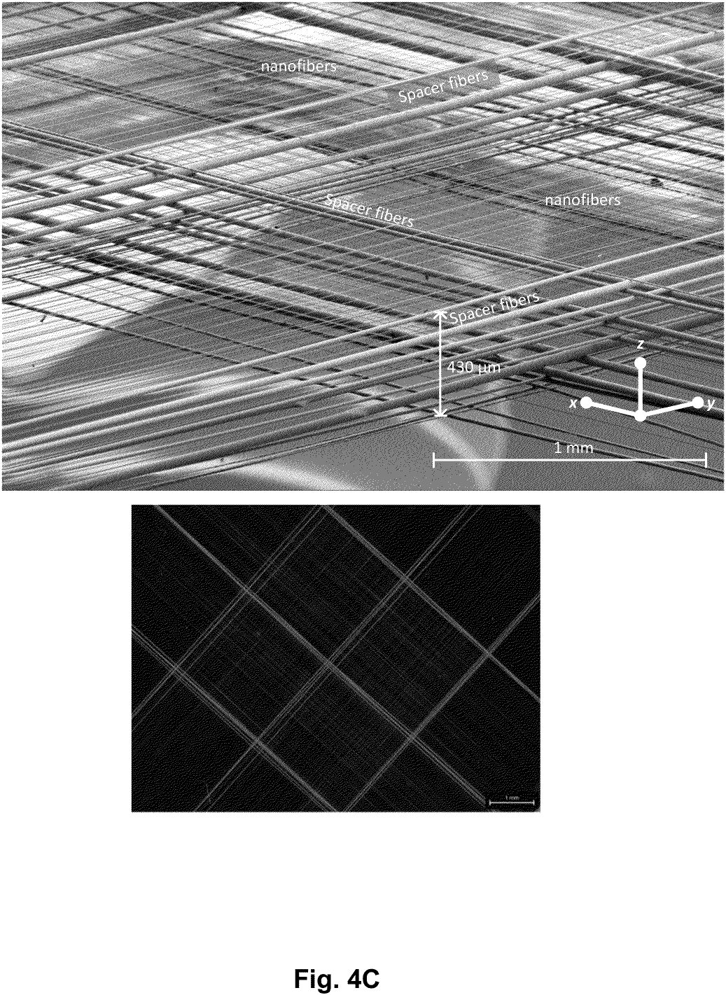

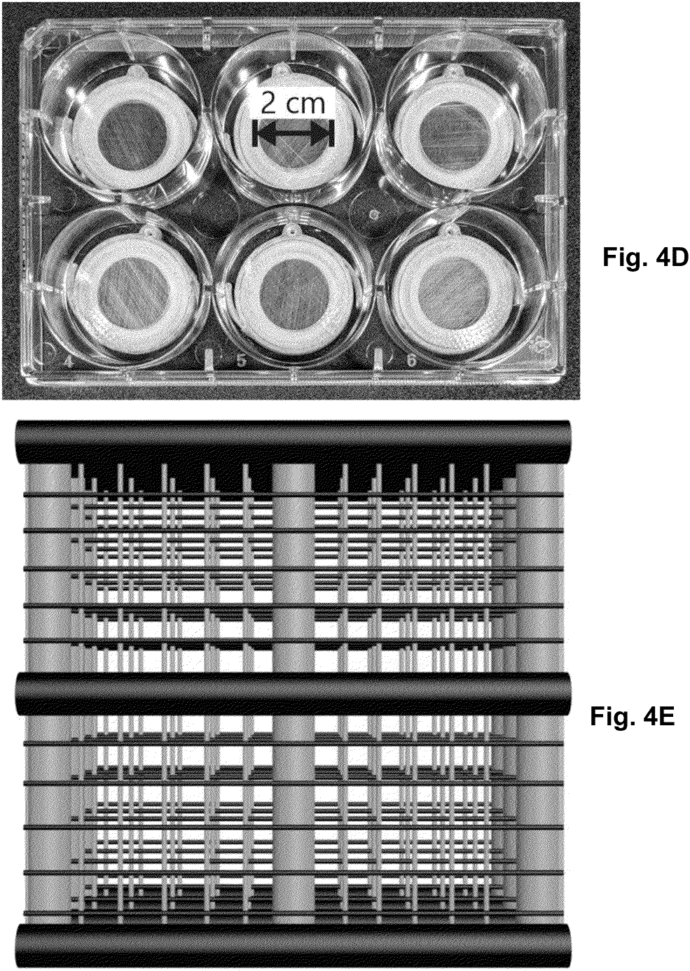

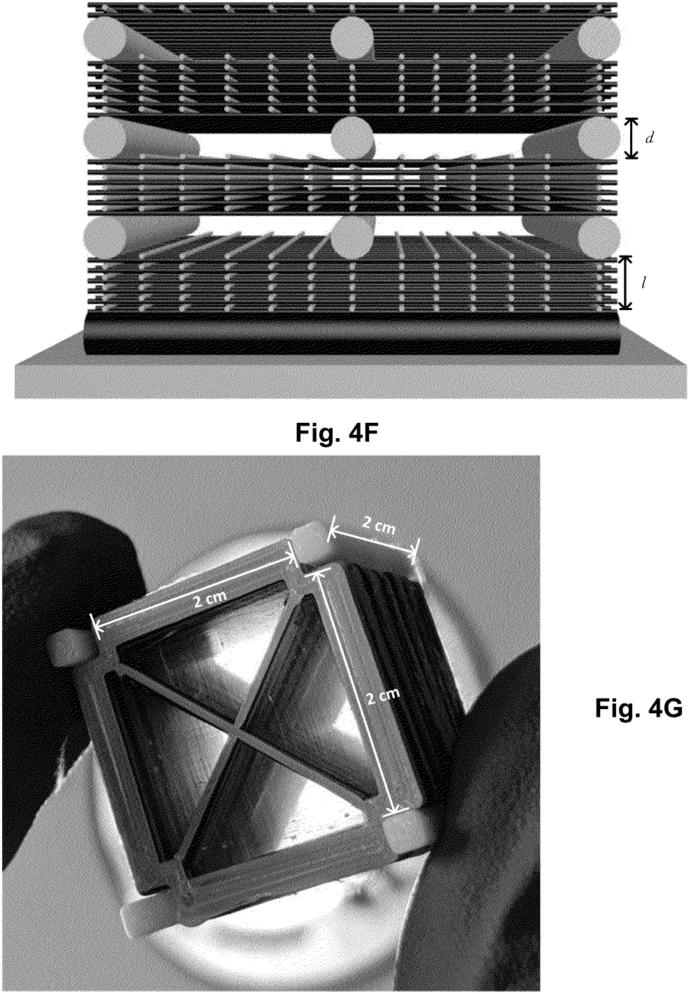

Large diameter fibers are used as spacer fibers to create the gap between nanofiber arrays in 3D scaffold. For example, see FIGS. 4C and 4F. Thousands of nanofibers (e.g., <2 um) can be included in a single array, however only a few spacer fibers (e.g., 3 or 4) are located between every two nanofiber layers in a 2 cm² area. The large diameter spacer fibers do not change the overall space available for the cell growth. However, if all of the nanofibers were large diameter fibers (e.g., 10 µm) were used with the same fiber spacing of 50 µm, the overall weight/volume fraction of fibers in a 3D structure would increase significantly. In the past decade, tissue engineering research has demonstrated that fibers below 2 µm are preferred for adherent cell anchoring and proliferation.

In some embodiments, the arrays comprise high-density fiber sections and low-density fiber sections, such that the layered arrays form cell growth areas (higher-density of fibers) and media transport areas (lower density of fibers) in the 3D scaffold. In some embodiments, multiple scaffolds can be stacked in multiple directions, e.g., like bricks or towers, to form larger 3D scaffolds without sacrificing stability.

In some embodiments, the nanofibers, spacer fibers, or spacer sheets can include a biocompatible material. In some embodiments, the nanofibers, spacer fibers, or spacer sheets can be edible or can include edible materials (e.g. polysaccharides such as chitosan, alginate, agar, pectin, lignin, carrageenan, xanthan, guar gums, cellulose etc. or proteins like casein, prolamin, prolamine, gelatin, soy, collagen, zein etc.).

In some embodiments, the fibers and/or other scaffolding materials can be formed from biocompatible materials such as polyethylene (PE), polyvinyl alcohol (PVA), polycaprolactone (PCL), polyacrylonitrile (PAN), and combinations thereof. Other biocompatible materials can be included, as can be appreciated by one of ordinary skill in the art. In other embodiments, the fibers and/or other scaffolding materials can be formed from edible materials as described above.

Methods for forming scaffolds - The present disclosure provides for methods of forming nanofiber scaffolds. Each nanofiber can be spaced and aligned in a predetermined, non-random manner, such that the resulting array of nanofibers is ordered and precisely spaced according to a particular need. For example, the distance between a pair of nanofibers is at a set distance (e.g., 10 µm to 100 µm, 10 µm to 20 µm) and/or at a desired angle (e.g., 0 to 179 degrees) relative to each other or to another fiber in the array. The fibers can be parallel to one another and/or in any desired angle and position relative to the first single filament nanofiber. In some embodiments, all of the nanofibers in a given array will all be parallel to one another. In some embodiments, the fibers can be placed at predetermined angles relative to one another or to the collection frame. In some embodiments, the fibers can be collected (e.g. non-randomly placed on the collection frame) such that they form crisscross or woven patterns in a given array.

The ordered arrays of nanofibers arrays can then be layered to form three-dimensional scaffolds that can be customized with varying fiber materials, diameters, elastic modulus, orientations, angles, spacing, spaces between layers, and geometries (e.g. flat sheet, tube, cylinder, cube, etc.).

In some embodiments, a long single filament nanofiber (e.g. a meter long) can be drawn and spiralled around a spacer frame with controlled motion of a stage to form an array or multiple arrays.

In various embodiments, multiple layers of 2D arrays can be formed into a 3D scaffold in an additive fabrication approach, and the layers can optionally be separated by spacer fibers. These spacer fibers can be formed using similar methods as the nanofibers. The spacer fibers can be formed from the same or different polymer solution from the nanofibers. The spacer fibers can be deposited from the same or a different nozzle on a fiber forming device than the nanofibers. Spacer fibers can be nanofibers or microfibers. In some embodiments, the spacer fibers can be about 1 µm to 100 µm in diameter. In various embodiments, the spacer fibers can comprise soluble or insoluble polymers. Soluble polymers, such as polyethylene oxide and polyvinyl alcohol, can be used when it is desirable for the spacer fibers to be sacrificial (e.g., not needed after formation of the 3D scaffold). Insoluble polymers, such as zein, polycaprolactone, polystyrene or polypropylene, can be used when the spacer fibers will not be removed after formation of the 3D scaffold. In some embodiments, the spacer fibers can be formed separately from the nanofibers (e.g., by 3D printing) and introduced as a layer between nanofiber array layers. In other embodiments, the spacer fibers can be formed using the same device used to form the nanofibers. In other embodiments, spacers could be ultra-thin films made of same material as the fibers. The ultra-thin film (10 um - 100 um thickness) can be cut into the desired shape using a blade or by laser cutting.

In some embodiments, at least two spacer fibers can be deposited on a 2D array. In some embodiments, more than one spacer fiber can be stacked to form a thicker layer. Multiple layers of spacer fibers can be laid between nanofiber array layers. The orientation of the spacer fibers can be the same as the nanofibers, can be placed orthogonally, or placed at another angle.

A second 2D array layer can be formed as described above and stacked on the spacer fiber layer to form a 3D scaffold. For example, a 2D nanofiber array forms a first layer having nanofibers in the x-y plane, followed by a spacer layer having fibers in the x-y plane, followed by a second 2D array layer having nanofibers in the X-Y plane. By alternately forming and collecting additional spacer fiber layers and 2D array layers in the X-Y plane, a 3D scaffold extending in the z-direction is formed. While the nanofibers and spacer fibers are all oriented in an x-y plane, each spacer fiber layer or 2D array layer can be rotated in the X-Y plane relative to a fiber orientation in the first 2D array layer. In this way, complicated, tunable patterns can be formed beyond parallel and orthogonal fiber layers.

In various embodiments, each nanofiber can independently have a diameter of about 50 nm - 100 µm, or about 100 nm to 5 µm. For example, all of the fibers can have the same diameter, all of the fibers oriented in one direction can have a first diameter and all of the fibers oriented in another direction can have a second diameter, or various patterns of diameters can be formed, such as alternating diameters or other patterns according to a desired outcome.

In various embodiments, the distance between any two nanofibers in a 2D array layer can be about 10 µm or greater, or about 10 µm to 1000 µm. In some embodiments, the distance between any two nanofibers in a 2D array layer can be more than 1000 µm. In various embodiments, the fiber orientation between any two layers of fibers can be between 0° and 179°. In various embodiments, the fiber orientation between any two fibers within a layer can be between 0° and 179°. Depending upon the desired properties of the scaffold (e.g. mechanical strength or ability to allow cell infiltration) individual nanofibers in the scaffold can have the same or different diameters from one another, and individual nanofibers can have the same or different compositions.

Advantageously, the porosity of the layers and scaffolds can be tuned to meet specific purposes. In various embodiments, the scaffold can have a porosity of about 20% to 99%, about 50% to 99%, or about 90% to 99%. The porosity of the layers and scaffolds can be tuned by adjusting the diameter of nanofibers, the spacing between nanofibers, the spacing and/or diameter of the spacer fibers, the thickness or number of spacer layers, and the orientation of spacer fibers and/or nanofibers.

In some embodiments, the spacer fibers can be removed or dissolved once the 3D scaffold has been formed. Such sacrificial fibers can be used in scaffold geometries where large inner spaces are desired, such as tubes or cylinders. Sacrificial spacer fibers can also be useful in scaffolds used to mimic complex vascular tissue or scaffolds used for 3D cell culturing.

In some embodiments, the nanofibers can be formed via touch spinning (see US 11,001,943, which is incorporated herein by reference in its entirety). In some embodiments, the nanofibers can be formed via gravitational drawing (see US 2021-02146575-A1, which is incorporated herein by reference in its entirety). Other fiber formation methods can include such as wet-spinning (see such as EP288782, ⁷, ⁸). In some embodiments, the nanofiber array layers can be mats.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

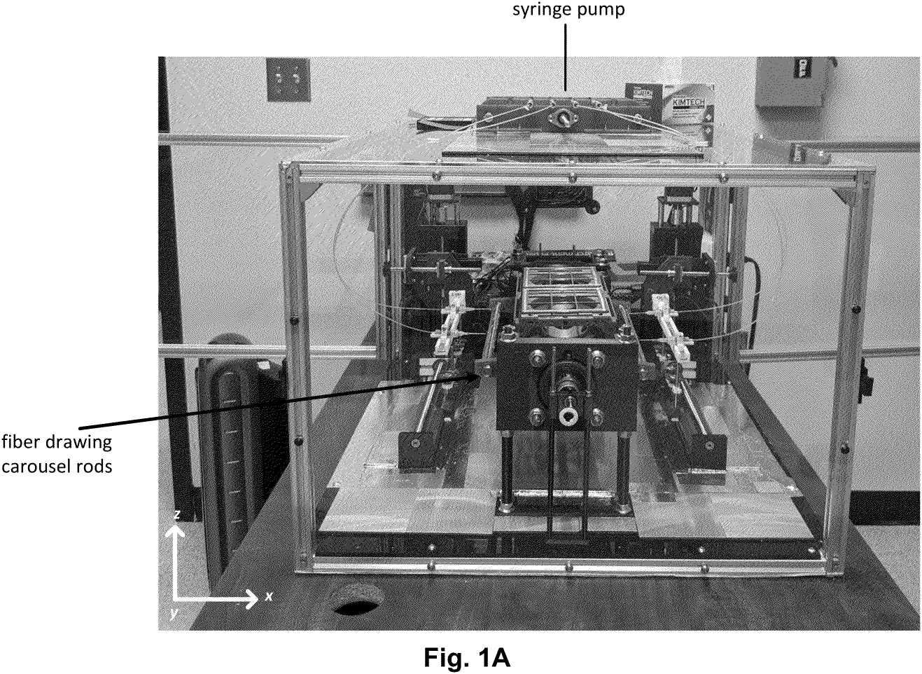

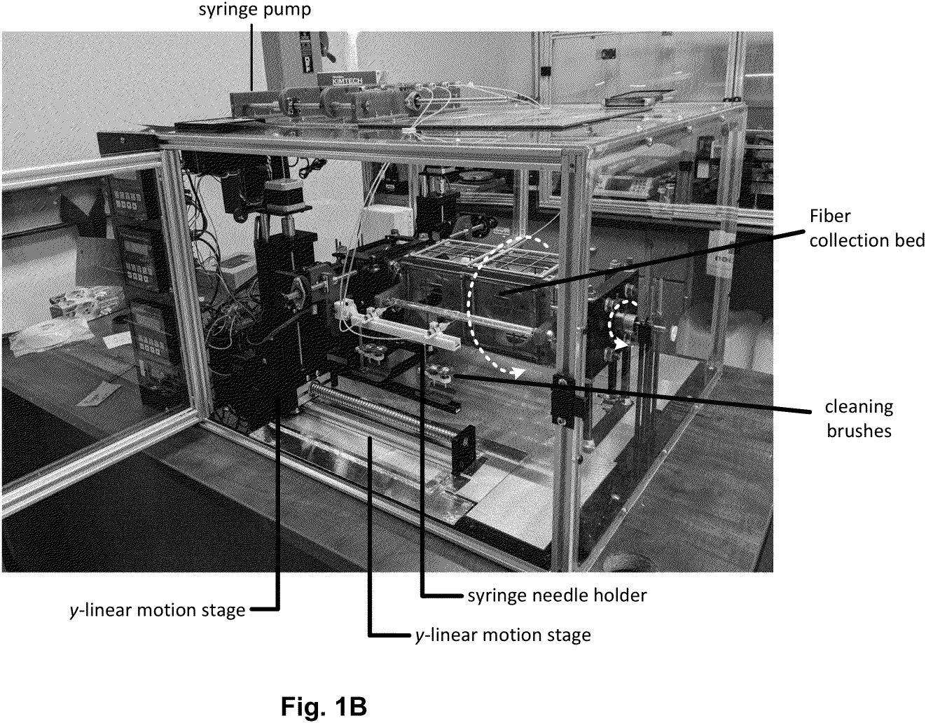

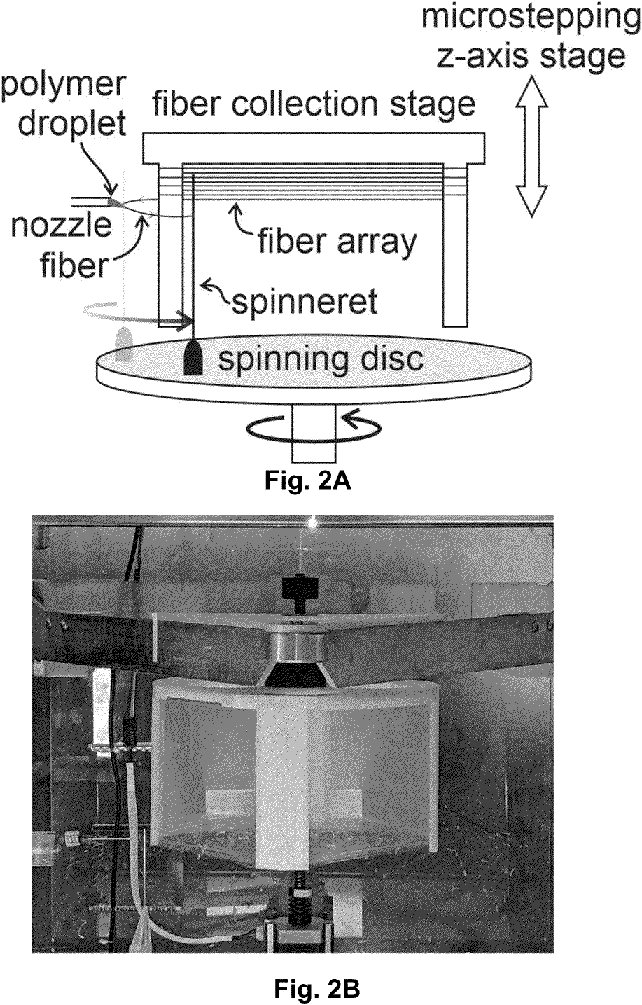

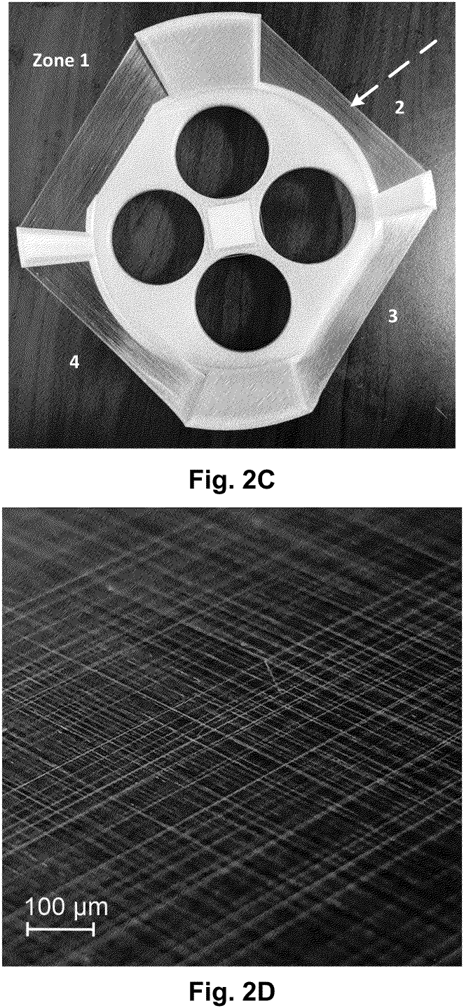

Provided are nanofiber scaffolding technologies and inserts for multi-well cell culture plates as single and scalable platform for rapid screening of adherent three-dimensional cell culture (3DCC) conditions scaled-up cell manufacturing for regenerative medicine (RM) and cultured meat (CM) applications. The three-dimensional nanofiber scaffolds (3DFS) based 3D cell culture (3DCC platform offers engineered scaffolds with biochemical cues, porosity (>90%), aspect ratio, unique scaffold design for controlled transport of media, and convenient adaptation for static and dynamic culture systems. These features, facilitated by fiber-spinning or fiber-fabrication technologies (examples shown in FIGS. 1A, 1B, and 2A, 2B), allow users to grow and harvest adherent cells from fibers conveniently for RM applications or utilize the whole scaffold as is for further applications in CM.

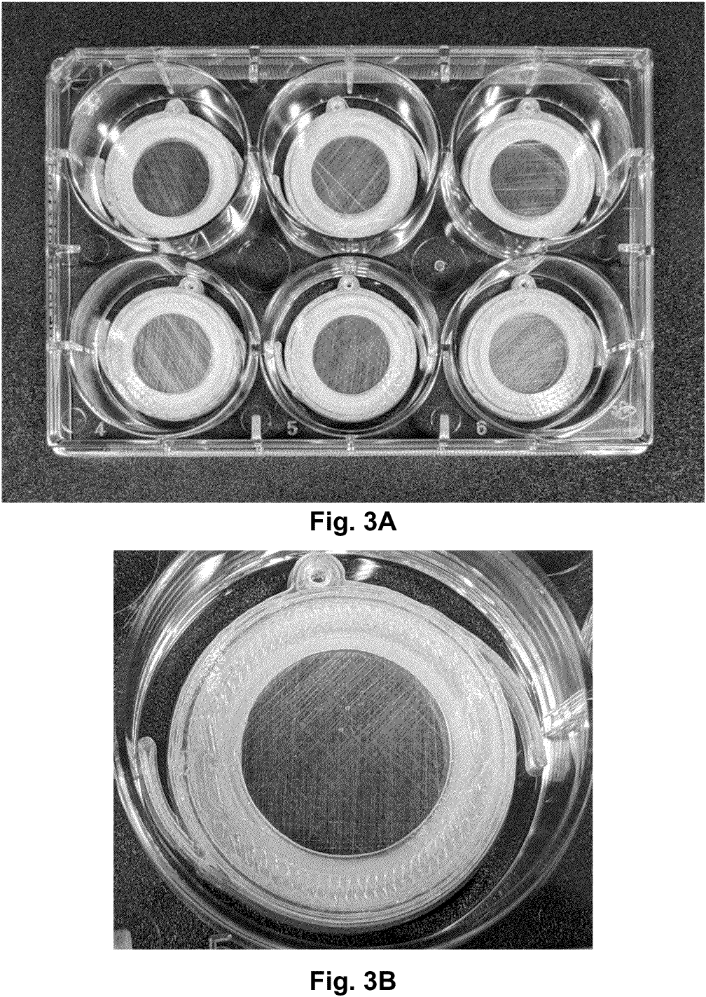

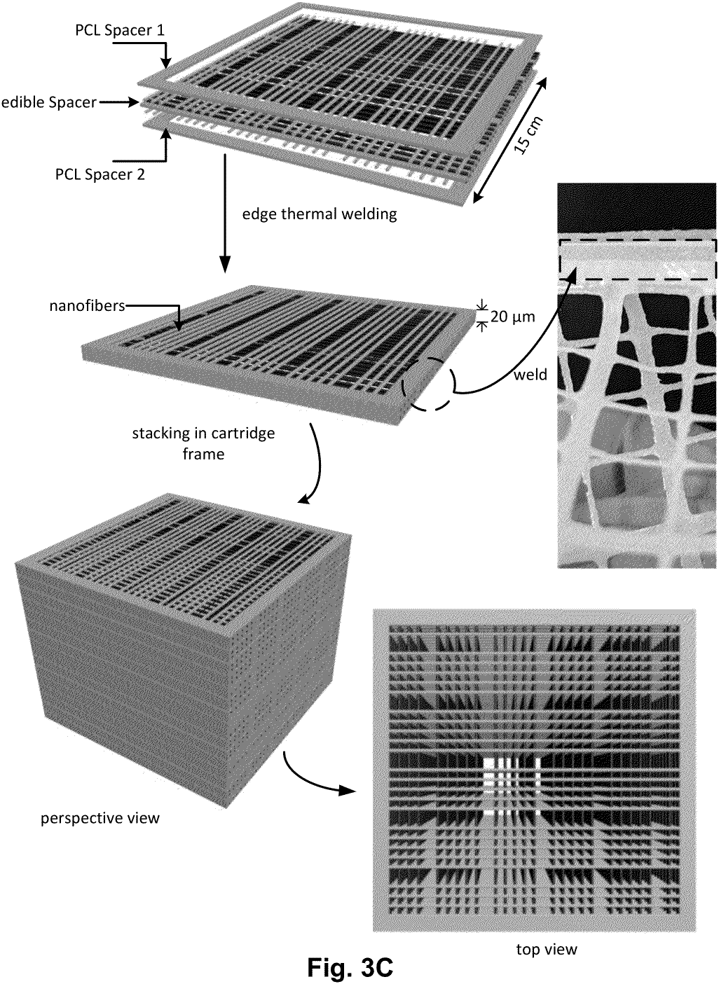

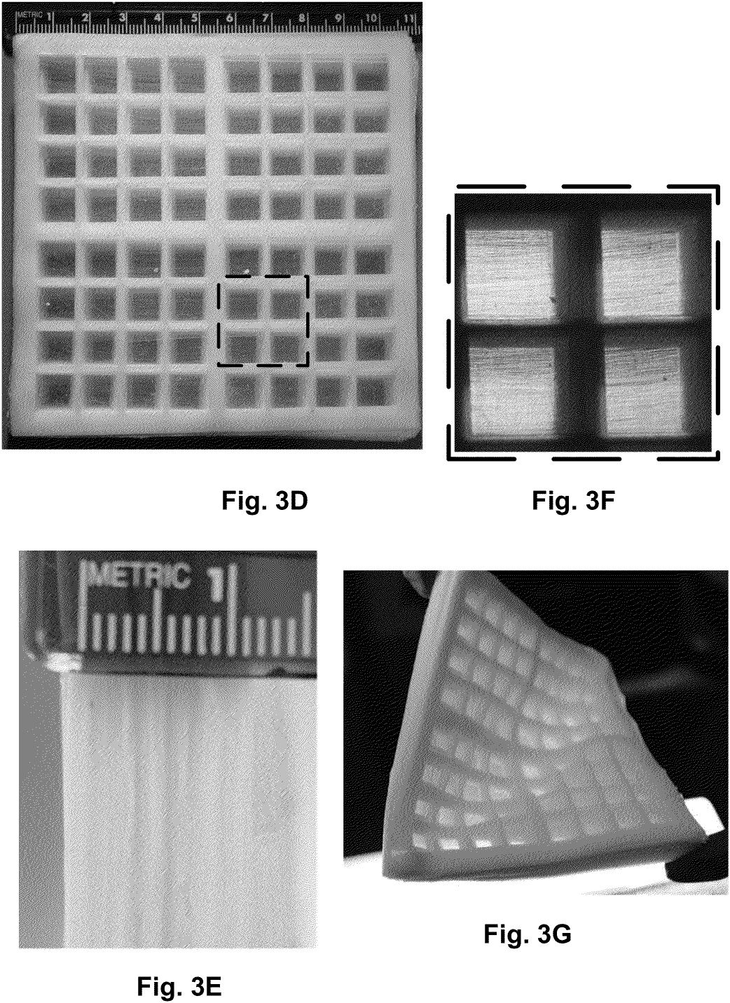

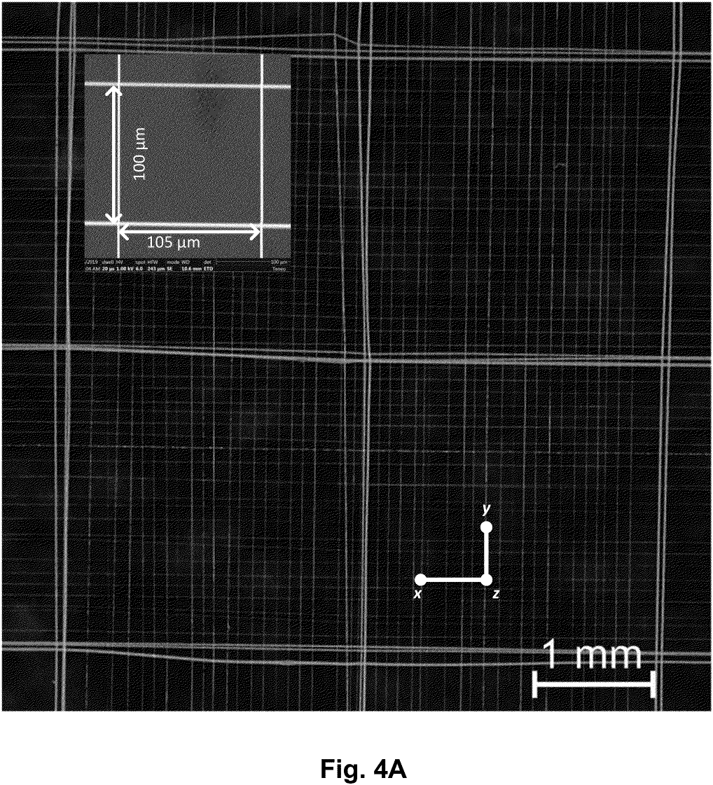

RM and CM customers need scalable 3DFS made of cell specific scaffolding materials. For example, cultured meat and seafood companies prefer fibers and 3DFS made of FDA approved edible materials, and RM companies seek biocompatible PCL-like materials. Combined with a universal mechanical fiber drawing process, such as gravitational drawing or touch spinning, 3DFS inserts embedded in multi-well plates can be rapidly fabricated for quick customization of cell culture parameters, and later these customized fiber scaffolds can be scaled quickly up to 5000 cm³/scaffold for scaled-up adherent cell manufacturing as presented in FIGS. 3A and 3C.

Automated and commercially scalable prototypes of touch spun based fiber fabrication and scaffolding device (FIGS. 1A, 1B, and 2A, 2B) are designed based on a spinneret rotating around a fiber collection bed/drum. These prototype devices have a built-in syringe pump for up to 6 polymer solution extruding channels, a fiber collection bed, an automated controllar set for xyz-axis linear motion stages, precision back and forth moving syring needle adapter stage, and cleaning brushes. Several accessories including a mechanical press for 3DFS embedding in multi-well plate inserts have been designed and tested. Several well plate inserts (FIGS. 3A and 3B) have been designed to use less plastic materials and consume less space in a well plate than a 3DFS. The inserts can house the 3D nanofiber scaffold and increase the ease of handling the 3DCC in and out of well plates. The prototype device in FIGS. 1A and 1B is capable of producing large area multi-layered 3D scaffolds (10×20×1 cm³) sufficient to generate 70 - 280 multi-well plate inserts from a single process of 6 h. This productivity can be ramped up to thousands of 3DFS inserts in singple process (<6 hours) for multiwell plates by increasing the length of the fiber fabrication carousel at commercial scale. In some embodiments, such as shown in FIG. 2C, zones are indicated for fiber diameter analysis as the fiber is continuously draw around the drum/collection frame. Fibers are aligned in one direction as each fiber is spun around the drum. As each turn of the spinneret completes, the drum is made to move up/down by set micrometers to achieve the fiber-fiber distance. To achieve the two direction alignment of fibers, fibers from zone 1 are collected on a frame first. The frame is turned 90 degrees and the fibers are then collected from zone 2 on top as part of the additive manufacturing scaffolding process discussed in FIG. 3C.

3DFS geometry. A gravitational fiber drawing (GFD; US 2021-0246575-A1) ^{3,4} method can consistently fabricate single filament nanofibers with a controlled fiber length⁴ (e.g.., about 0.01 m to 2 m) with an adjustable, broad range of fiber diameters (e.g., from 0.05 nm to 100 µm). The devices, integrated with GFD technique, employ ultra-long individual nanofibers as building blocks to fabricate precision-aligned 2D nanofiber arrays. These 2D nanofiber arrays are stacked in a layer-by-layer fashion to build a 3D scaffold in an additive manufacturing approach to achieve any desired geometry (e.g., cube, cylinder, etc). The GFD technique is convenient for rapid screening of cell culture parameters at research scale.

A touch spinning device (U.S. Pat. 11,136,695, U.S. Pat. 11,001,943, and FIGS. 1A, 1B) can be modified to produce rapid scaffolds of any size with less precision for cell culture parameter optimization and scaled-up cell manufacturing based on cell-specific application demands. The devices in the above-referenced patents can form mats and/or arrays of single filament fibers that can be stacked or folded into layers.

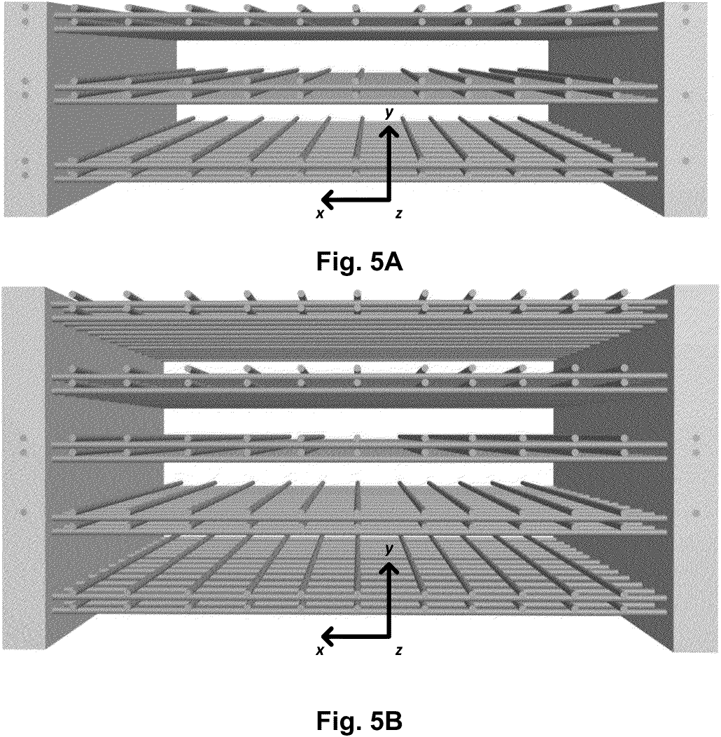

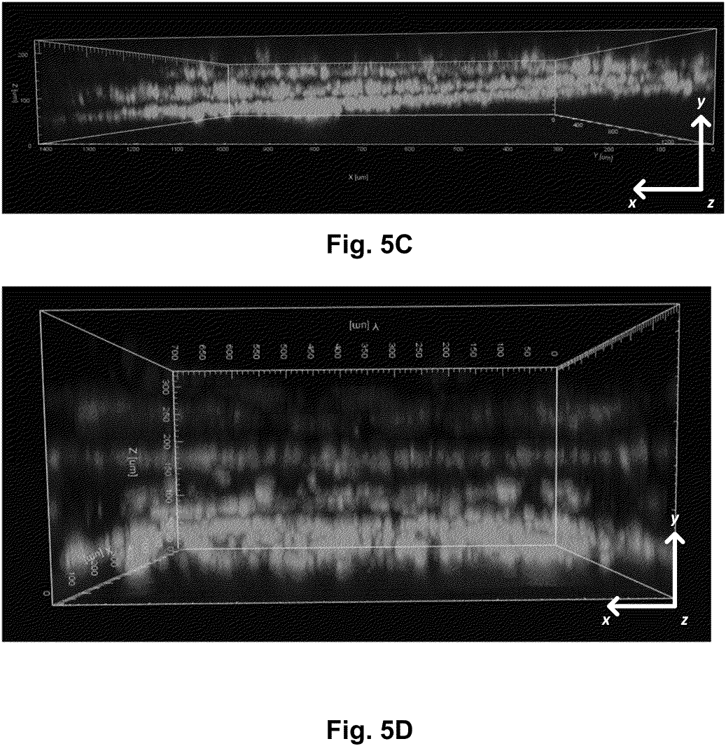

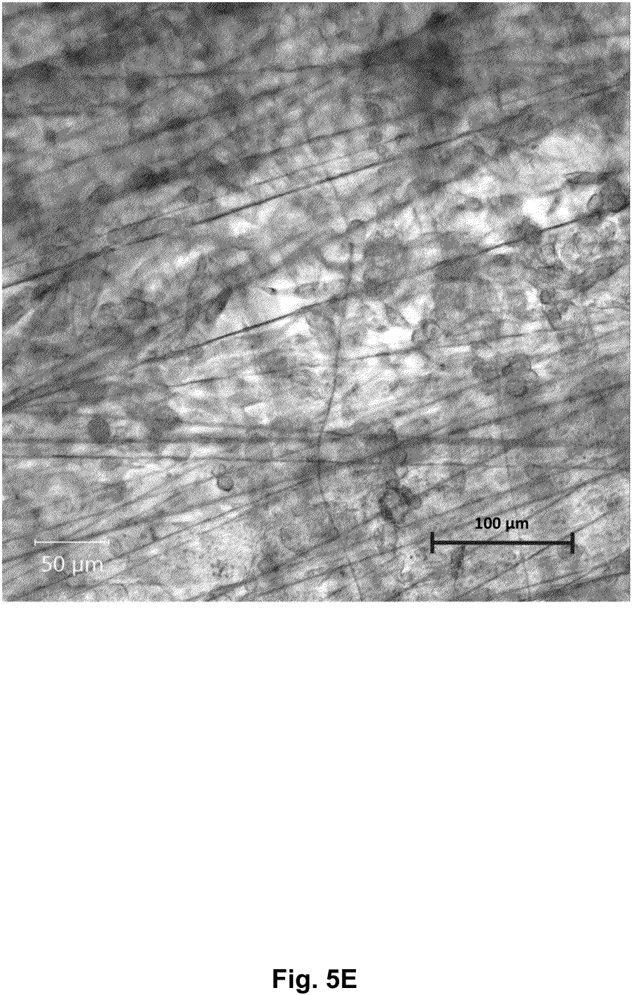

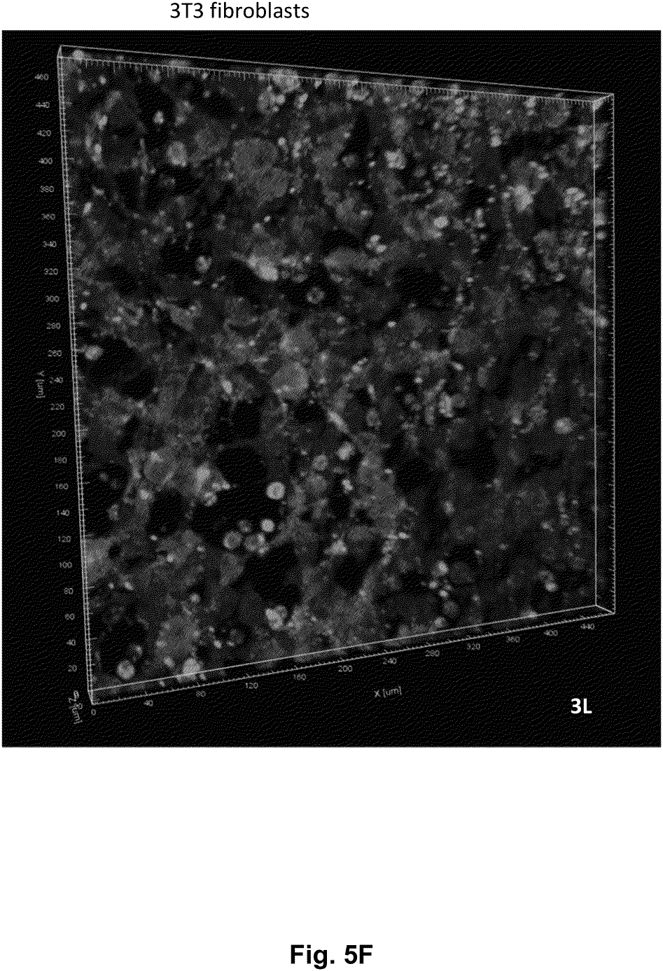

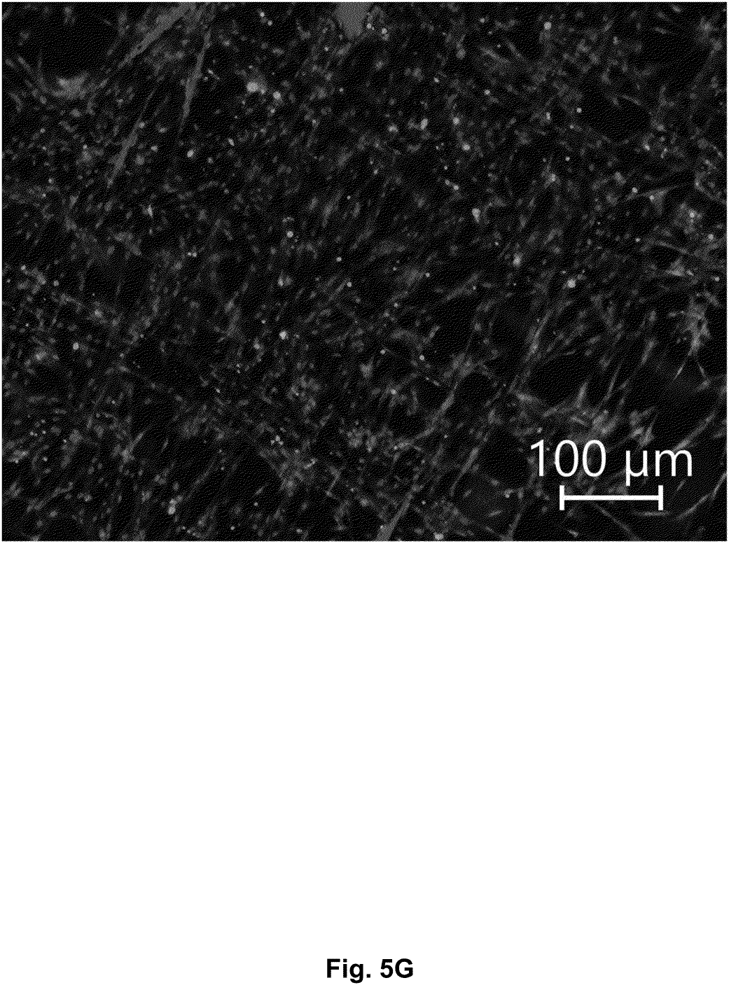

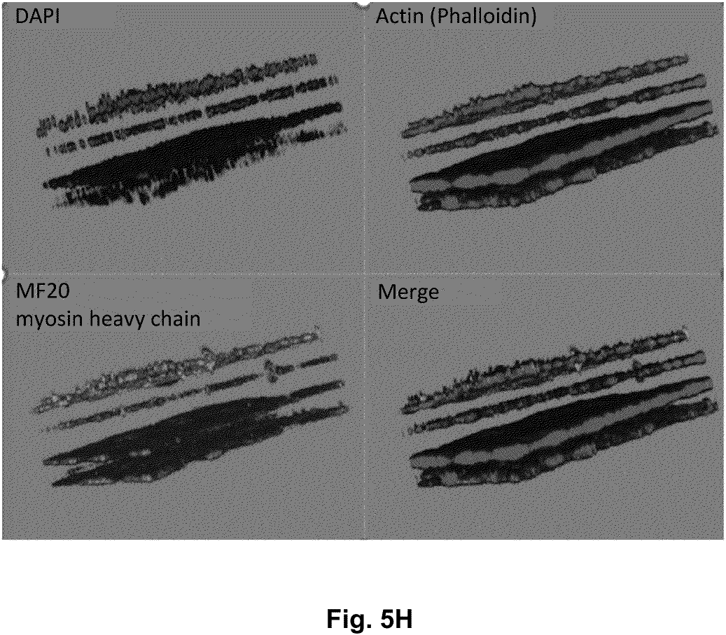

Porosity and Vasculature: We have demonstrated ability to tune the porosity and “vasculature” (microchannels in the 3D scaffolds) to a high degree of precision through the 3DFS design as shown in FIGS. 4E, 4F, 5A and 5B. Varying the fiber spacing and layer spacing resulted in controlled porosity of 3DFS from 50% up to 95% for high density cell culture. The 3DFS with 95% porosity remained stable during the media exchanges and culture growth for over two weeks in static 3DCC studies. Vasculature is also tested by varying the layer spacing where cells are anchored and proliferated in their own restricted/confined zones (layers) as shown in FIGS. 5D and 5E. Cell phenotype variations (FIG. 5F) are observed depending on the parameters of the nanofiber layers parameters and the culture duration.

Biochemical and Biomechanical cues: In our preliminary work, the PCL fibers are blended with poly(glycidyl methacrylate) to leverage the amino-epoxy chemistry. The lysine amino acids in proteins and growth factors could be reacted spontaneously with epoxide groups exposed on fiber surface, thus anchoring/coating the proteins on fiber surface. The protein decoration on the fiber scaffold is observed to increase the 3T3 fibroblast growth as presented in FIGS. 5C-5F. We have also demonstrated^{5,6} the tunability of fiber biomechanical properties in a Young’s modulus range of 100 KPa - 400 MPa.

3D cell culture studies: 3DFS inserts in multi-well plates are tested with different adherent cell cultures in static culture conditions with periodic media exchange. These cell cultures are observed to anchor and grow specifically on nanofibers compared to spacer microfibers. Thus, fiber diameter is a critical parameter for scaffold engineering. Further, micro-perfusion channels are also tested as shown in FIG. 5A and FIG. 5B with a controlled gap between multiple nanofiber layers. In a first case (FIGS. 5C-5F), PCL nanofiber scaffolds are seeded with few thousand cells of 3T3 fibroblasts. These fibroblasts continue to anchor, extend/elongate (FIG. 5E), and grow along fibers in 3D with periodic media exchange for up to 5 weeks.

Cultured meat application: In a proof-of-concept work, Bovine satellite cells (BSCs) (precursor beef cells) developed by the Tufts University research team (Dr. David Kaplan’s group) was tested on our 3D fiber scaffold insert in multi-well plates (FIGS. 5G-5H). Dense layers of BSCs were developed in the confined nanofiber layers separated by the micro-perfusion channels in the 3D structure. The cells were further induced to differentiate towards the muscle cells.

Large 3DFS fabrication: The additive manufacturing process with nanofibers can be scaled to fabricate 3DFS with volumes from 0.125 cm³ up to 5000 cm³ per scaffold. Currently, our scaffolding device prototypes are semiautomated with a maximum scaffold area of 20×10 cm². The time required to fabricate a precision 2D nanofiber array of 1×1 cm² or 11×11 cm² is nearly the same. These 2D arrays can be stacked together with spacer layers between them to build large 3DFS cartridges up to 5000 cm³/scaffold. The spacer layers can be fabricated using microfibers (diameter up to 50 µm, FIGS. 4A-4F) along with nanofibers in single continuous process or can be 3D printed spacer sheets for thicknesses >50 µm (FIG. 3C) as needed for a particular vasculature design. To demonstrate the geometry and linear scalability, we have constructed a 3DFS cube of 8 cm³ (FIG. 4G) and 121 cm³ (FIG. 3D) using a semiautomated prototype scaffolding devices (FIGS. 1A, 1B and 2A, 2B). However, the 3DFS fabrication process is not limited to these designs or geometries. The 8 cm³ cube can be fabricated in under 4 h, consisting of 48 spacer layers and 96 layers of nanofiber 2D arrays. Similarly, 121 cm³ can be fabricated in under 12 h with 90 spacer sheets (50 µm thick) and 90 nanofiber layers. With an automated scaffolding device of the next generation and by using prefabricated spacer sheets, we have estimated that a 5000 cm³ (e.g., cube side of 17 cm) 3DFS is feasible to manufacture in less than 3 h. The productivity could be improved further with multiplication of scaffolding modules so that multiple scaffolds could be produced in parallel. The area occupied by one module is about 7 square feet; a 200 square feet room could accommodate 60 modules arranged on three-level stages to manufacture about 120 of 5000 cm³ cubes in one shift.

Spacer thermal welding: Another improvement for 3D scaffold fabrication in an additive manufacturing approach and the 3D scaffold-based adherent cell culture scale-up is thermal welding of nanofiber layers between spacer sheets as shown in FIGS. 3C and 3D. We have recently developed a process for thermal welding of 50 µm thick PCL spacer sheets with nanofiber arrays embedded between them (FIG. 3C). This process could be used to weld pieces up to 20 cm in linear dimension (FIG. 3D shows 11 cm). Nanofiber 2D arrays stacked between spacer sheets made of edible materials could be welded between sacrificial PCL spacer films as shown in FIG. 3C. Each welded stack of nanofibers and spacers will be around 200 µm thick suitable for 3D cell cultures. The PCL spacer sheets can be replaced with edible polymer films (e.g., prolamin proteins) to be compatible with edible applications while being suitable for thermal welding. These stacks of welded fiber layers can be further positioned in a biocompatible or food compatible adapter frame (cartridge) to stabilize the large 3DFS (e.g., > 300 cm³ or a scaffold with length 10 cm, width 15 cm and thickness 2 cm, similar to a whole-cut beef) stack through the welding process used to create flexible 3DFS (FIG. 3G). The cartridge geometry can be designed to be a cube, rectangle, cylinder, etc. The whole cartridge can be placed as is in a dynamic culture vessel for cell expansion. Post culture, these stacks can be separated for cell harvesting, or used as whole cut meat, excluding the sacrificial PCL/spacer boundaries. The spacer welding technology is widely applicable for rapid multi-well plate insert manufacturing and large scaffold fabrication.

The PCL welding process can also be replaced with biocompatible glue between layers depending on compatibility with scaffold materials and cell toxicity.

3DFS Sterilization: PCL or other material based 3DFS are tested for sterilization using ethanol, UV, and ethylene oxide. Commercially viable sterilization techniques for edible scaffolds can also be used as can be envisioned by one of ordinary skill in the art.

### REFERENCES

1 Tokarev, A., Trotsenko, O., Asheghali, D. & Minko, S. Nanofiber spinning apparatus and method. U.S. Pat. U.S. Utility Pat. No. 11001943 (will be issued on May 11, 2021 (received confirmation from USPTO)).

2 Sharma, S., Asheghali, D., Minko, S., Yadavalli, N. S. & Gao, H. Methods and devices for multi-layer nanofibers. Utility Patent App. 20190352803A1 United States Patent (2019).

3 Yadavalli, N. S., Asheghali, D., Tokarev, A. & Minko, S. Methods and devices for making nanofibers and nanofiber scaffolds. U. S. Utility Patent 17/169,940, Feb. 8, 2021.

4 Yadavalli, N. S. et al. Gravity Drawing of Micro- and Nanofibers for Additive Manufacturing of Well-Organized 3D-Nanostructured Scaffolds. Small 16, e1907422, doi:10.1002/smll.201907422 (2020).

5 Lee, S.-J. et al. Touch-Spun Nanofibers for Nerve Regeneration. ACS Applied Materials & Interfaces 12, 2067-2075, doi:10.102⅟acsami.9b18614 (2020).

6 Asheghali, D. et al. Enhanced neuronal differentiation of neural stem cells with mechanically enhanced touch-spun nanofibrous scaffolds. Nanomedicine 24, 102152, doi:10.1016/j.nano.2020.102152 (2020).

7. Pati et. al Development of Ultrafine Chitosan Fibers Through Modified Wetspinning Technique. Journal of Applied Polymer Science, Vol. 121, 1550-1557 (2011).

8. Hirano, S. Wet-Spinning and Applications of Functional Fibers Based on Chitin and Chitosan. Macromol. Symp. 2001, 168, 21-30.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt% to about 5 wt%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

What is claimed is:

1. A 3D cell culture scaffold, comprising:
a plurality of laminated nanofiber layers, wherein each layer is formed by an array of nanofibers, wherein each of the nanofibers in the arrays has a predetermined diameter of about 50 nm to 100 µm;
wherein the nanofibers comprise a natural polymer, a synthetic polymer, a biocompatible material, or a combination thereof;
wherein the nanofibers in the arrays have controlled alignment, angle, and spacing from one another; and
wherein 3D cell culture the scaffold has a porosity of 50% to 99%.

2. The 3D cell culture scaffold according to claim 1, wherein the nanofiber layers are laminated together by thermal welding or by biocompatible glue.

3. The 3D cell culture scaffold according to claim 1, wherein the nanofiber layers are spaced apart from one another by a plurality of spacer fibers or spacer sheets such that the spacer fibers or the spacer sheets form an intermediate layer between the nanofiber layers.

4. The 3D cell culture scaffold according to claim 3, wherein the plurality of spacer fibers or spacer sheets are sacrificial.

5. The 3D cell culture scaffold according to claim 4, wherein the plurality of spacer fibers or spacer sheets comprise polyethylene oxide and polyvinyl alcohol.

6. The 3D cell culture scaffold according to claim 3, wherein the plurality of spacer fibers or spacer sheets comprise an insoluble polymer selected from zein, polycaprolactone, polystyrene, polypropylene, or a combination thereof.

7. The 3D cell culture scaffold of claim 1, wherein the arrays comprise high-density fiber sections and low-density fiber sections, such that the layered nanofiber arrays form cell growth areas and media transport areas in the scaffold.

8. The 3D cell culture scaffold of claim 1, wherein the scaffold has a volume of about 0.125 cm³ to 5000 cm³.

9. The 3D cell culture scaffold of claim 1, wherein the nanofibers are from about 100 nm to 5 µm in diameter.

10. The 3D cell culture scaffold of claim 1, wherein the layers are formed by stacked nanofiber arrays.

11. The 3D cell culture scaffold of claim 1, wherein the layers are formed by continuous arrays folded into layers.

12. The 3D cell culture scaffold of claim 1, wherein the scaffold is embedded in a well-plate insert.

13. An edible 3D scaffold for cultured meat, comprising:
a plurality of laminated nanofiber layers, wherein each layer is formed by an array of nanofibers, wherein each of the nanofibers in the arrays has a predetermined diameter of about 50 nm to 100 µm;
wherein the nanofibers are edible;
wherein the nanofibers in the arrays have controlled alignment, angle, and spacing from one another; and
wherein the scaffold has a porosity of 50% to 99%.

14. The edible 3D scaffold according to claim 13, further comprising edible spacer fibers or edible spacer sheets forming an intermediate layer between each of the nanofiber layers.

15. The edible 3D scaffold according to claim 14, wherein the edible nanofibers are comprised of polyvinyl acetate, cellulose, methyl cellulose, cellulose acetate, chitosan, alginic acid, soy proteins, prolamine proteins, collagen, silk fibroin, chitosan, alginate, agar, pectin, lignin, carrageenan, xanthan, guar gums, casein, gelatin, soy, zein, or combinations thereof.

16. The edible 3D scaffold according to claim 14, wherein the edible spacer fibers or spacer sheets are comprised of polyvinyl acetate, cellulose, methyl cellulose, cellulose acetate, chitosan, alginic acid, soy proteins, prolamine proteins, collagen, silk fibroin, chitosan, alginate, agar, pectin, lignin, carrageenan, xanthan, and guar gums, casein, gelatin, soy, zein, or combinations thereof.
